(19) United States
(12) Patent Application Publication
(10) Pub. No.: US 2007/0293791 A1
(43) Pub. Date: Dec. 20, 2007

(54) Guidewire With Lubricious Proximal Portion

(76) Inventors: Jeong Lee, Diamond Bar, CA (US); Aaron Baldwin, Newark, DE (US); Roseminda White, Wildomar, CA (US); Kristen M. Nolan, Murrieta, CA (US); Emmanuel C. Biagtan, Temecula, CA (US)

Correspondence Address: Fulwider Patton LLP, Howard Hughes Center, 6060 Center Drive, Tenth Floor, Los Angeles, CA, 90045, US

(21) Appl. No.: 11/424,819

(22) Filed: Jun. 16, 2006

(51) Int. Cl. A61M 25/09

(52) U.S. Cl. 600/585

(57) ABSTRACT

A guidewire has a proximal portion and a distal portion. The distal portion may be coated, as with a hydrophilic or other coating, or may be uncoated. The proximal portion, however, is coated with a specially lubricious coating that serves to reduce resistance and to prevent lock up as the guidewire moves through a guiding catheter. The proximal portion may, for example, be coated with a base layer, such as TEFLON® (polytetrafluoroethylene), and one or more top layers, such as a silicone-based layer. In one approach, two silicone-based top layers are applied. Alternatively, a top layer of spray PTFE, paraffins, olefins, and/or fats may be applied. As an alternative, the base layer may be optional, such that the top layer or layers is coated onto bare metal, or a base other than TEFLON® may be used.

### BACKGROUND OF THE INVENTION

This invention relates to the field of guidewires for advancing intraluminal devices such as stent delivery catheters, balloon dilatation catheters, atherectomy catheters and the like within body lumens.

In a typical coronary procedure a guiding catheter having a preformed distal tip is percutaneously introduced into a patient's peripheral artery, e.g. femoral or brachial artery, by means of a conventional Seldinger technique and advanced therein until the distal tip of the guiding catheter is seated in the ostium of a desired coronary artery. There are two basic techniques for advancing a guidewire into the desired location within the patient's coronary anatomy, the first is a preload technique which is used primarily for over-the-wire (OTW) devices and the bare wire technique which is used primarily for rail type systems.

With the preload technique, a guidewire is positioned within an inner lumen of an OTW device such as a dilatation catheter or stent delivery catheter with the distal tip of the guidewire just proximal to the distal tip of the catheter and then both are advanced through the guiding catheter to the distal end thereof. The guidewire is first advanced out of the distal end of the guiding catheter into the patient's coronary vasculature until the distal end of the guidewire crosses the arterial location where the interventional procedure is to be performed, e.g. a lesion to be dilated or a dilated region where a stent is to be deployed. The catheter, which is slidably mounted onto the guidewire, is advanced out of the guiding catheter into the patient's coronary anatomy over the previously introduced guidewire until the operative portion of the intravascular device, e.g. the balloon of a dilatation or a stent delivery catheter, is properly positioned across the arterial location.

Once the catheter is in position with the operative means located within the desired arterial location, the interventional procedure is performed. The catheter can then be removed from the patient over the guidewire. Usually, the guidewire is left in place for a period of time after the procedure is completed to ensure reaccess to the arterial location is it is necessary. For example, in the event of arterial blockage due to dissected lining collapse, a rapid exchange type perfusion balloon catheter such as described and claimed in U.S. Pat. No. 5,516,336 (McInnes et al), can be advanced over the in-place guidewire so that the balloon can be inflated to open up the arterial passageway and allow blood to perfuse through the distal section of the catheter to a distal location until the dissection is reattached to the arterial wall by natural healing.

With the bare wire technique, the guidewire is first advanced by itself through the guiding catheter until the distal tip of the guidewire extends beyond the arterial location where the procedure is to be performed. Then a rail type catheter, such as described in U.S. Pat. No. 5,061,395 (Yock) and the previously discussed McInnes et al. which are incorporated herein by reference, is mounted onto the proximal portion of the guidewire which extends out of the proximal end of the guiding catheter which is outside of the patient. The catheter is advanced over the catheter, while the position of the guidewire is fixed, until the operative means on the rail type catheter is disposed within the arterial location where the procedure is to be performed. After the procedure the intravascular device may be withdrawn from the patient over the guidewire or the guidewire advanced further within the coronary anatomy for an additional procedure.

Conventional guidewires for angioplasty, stent delivery, atherectomy and other vascular procedures usually comprise an elongated core member with one or more tapered sections near the distal end thereof and a flexible body such as a helical coil or a tubular body of polymeric material disposed about the distal portion of the core member. A shapable member, which may be the distal extremity of the core member or a separate shaping ribbon which is secured to the distal extremity of the core member extends through the flexible body and is secured to the distal end of the flexible body by soldering, brazing or welding which forms a rounded distal tip. Torquing means are provided on the proximal end of the core member to rotate, and thereby steer, the guidewire while it is being advanced through a patient's vascular system.

Further details of guidewires, and devices associated therewith for various interventional procedures can be found in U.S. Pat. No. 4,748,986 (Morrison et al.); U.S. Pat. No. 4,538,622 (Samson et al.): U.S. Pat. No. 5,135,503 (Abrams); U.S. Pat. No. 5,341,818 (Abrams et al.); U.S. Pat. No. 5,345,945 (Hodgson, et al.) and U.S. Pat. No. 5,636,641 (Fariabi) which are hereby incorporated herein in their entirety by reference thereto.

A problem can arise in practice, as the guidewire moves through the catheter and encounters clumps of clotted blood, dried contrast, or combinations thereof that adhere to the proximal section of the guide wire. The guidewire tends to meet movement resistance within the catheter, and may even lock up. While guidewire designs heretofore utilized in the prior art have been adequate, improvements have been sought to make guidewires less vulnerable to movement resistance and locking up. The present invention meets these and other needs.

### SUMMARY OF THE INVENTION

Briefly, and in general terms, the present invention provides a new and improved guidewire having a lubricious proximal portion. The lubricious proximal portion reduces resistance and/or lock up caused by interaction between the proximal portion and clotted blood, dried contrast, or combinations thereof that normally adhere to the proximal section of the guide wire.

Guidewires according to the present invention have a “proximal” portion and a “distal” portion. Typically, the “distal” portion is the relatively short region of the guidewire that extends from the tip. The “distal” portion typically travels outside of the guiding catheter during use, and into the targeted portion of the anatomy, thereby coming into contact with the arterial wall. On the other hand, the “proximal” portion of the guidewire is typically the lengthy segment extending immediately from the “distal” portion. In the “proximal” portion, unlike most “distal” portions, there is not a reduction in the diameter of the core of the wire.

In one embodiment of the invention, the proximal portion is coated with a base coat of a lubricious coating such as TEFLON®, although a variety of other base coats can be used. Then, at least one top coat of a different lubricious coating such as, for example, a silicone-based coating, is applied over the base coat. This combination of base coat and top coat imparts a lubricity that improves the ability of the guidewire to overcome resistance and prevent lock up during use.

In another embodiment, a guidewire has a proximal portion and a distal portion. The proximal portion is coated with a lubricious coating such as a silicone-based coating, a paraffin, an olefin, and/or a fat. In this embodiment, the lubricious coating may optionally be applied directly to bare metal, without a base coat, if desired. Alternatively, a base coat such as at least one of a fluoropolymer, polyethylene, and polypropylene may be first applied to the metal.

Another approach is to provide a guidewire in which the proximal portion is coated with a first base coat of lubricious coating. A top coat of a second, hydrophobic, lubricious coating is applied on the base coat. The distal portion, on the other hand, is coated with a hydrophilic coating. This combination of a hydrophobic, particularly lubricious coating on the proximal portion and a hydrophilic coating on the distal portion is particularly well-suited to the operation of the guidewire. The hydrophilic coating is advantageous on the distal portion of the guidewire that comes into contact with the arterial walls, whereas the hydrophobic, lubricious coating on the proximal portion prevents undesired resistance from blood clots and the like as the guidewire moves through the catheter.

Further advantages and details of embodiments of this invention will be apparent from the following more detailed description, when taken in conjunction with the accompanying drawings of exemplary embodiments.

### BRIEF DESCRIPTION OF THE DRAWINGS

FIG. 1 is an elevational view partially in section of a guidewire embodying features of the invention;

FIG. 2 is a transverse cross-sectional view of the guidewire shown in FIG. 1 taken along lines 2-2;

FIG. 3 is a transverse cross-sectional view of the guidewire shown in FIG. 1 taken along the lines 3-3;

FIG. 4 is an elevational view partially in section of an alternative guidewire embodiment;

FIG. 5 is an elevational view partially in section of another alternative guidewire embodiment;

FIG. 6 is an elevational view partially in section of another alternative guidewire embodiment;

FIG. 7 is a graph generally showing a force to displacement relationship of a guidewire that is not coated on the proximal portion, as it works it way through a test vascular system and ultimately locks up due to interaction with clots or the like; and

FIG. 8 is a graph generally showing a force to displacement relationship of a guidewire having a silicone-based coating atop a TEFLON® base coat, in which the guidewire does not locks up.

### DETAILED DESCRIPTION OF THE PREFERRED EMBODIMENTS

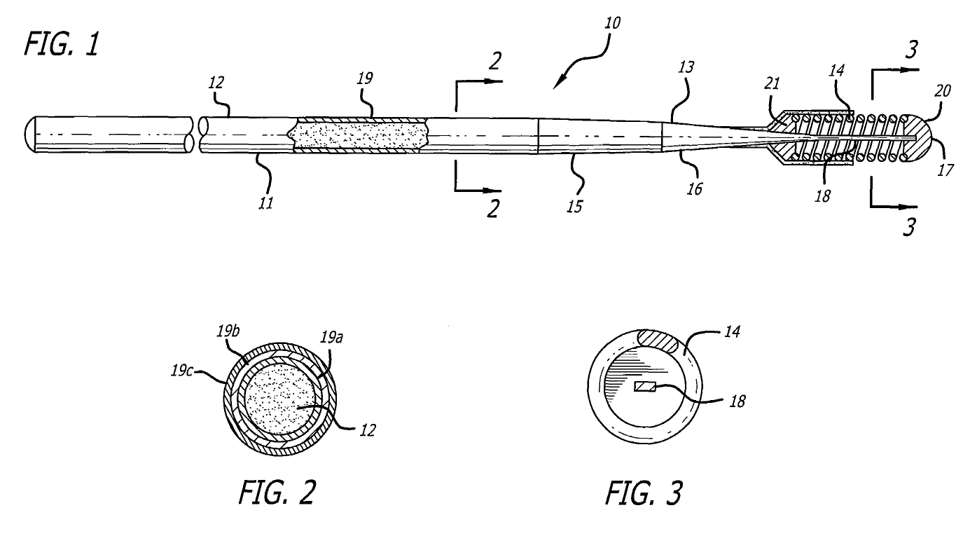

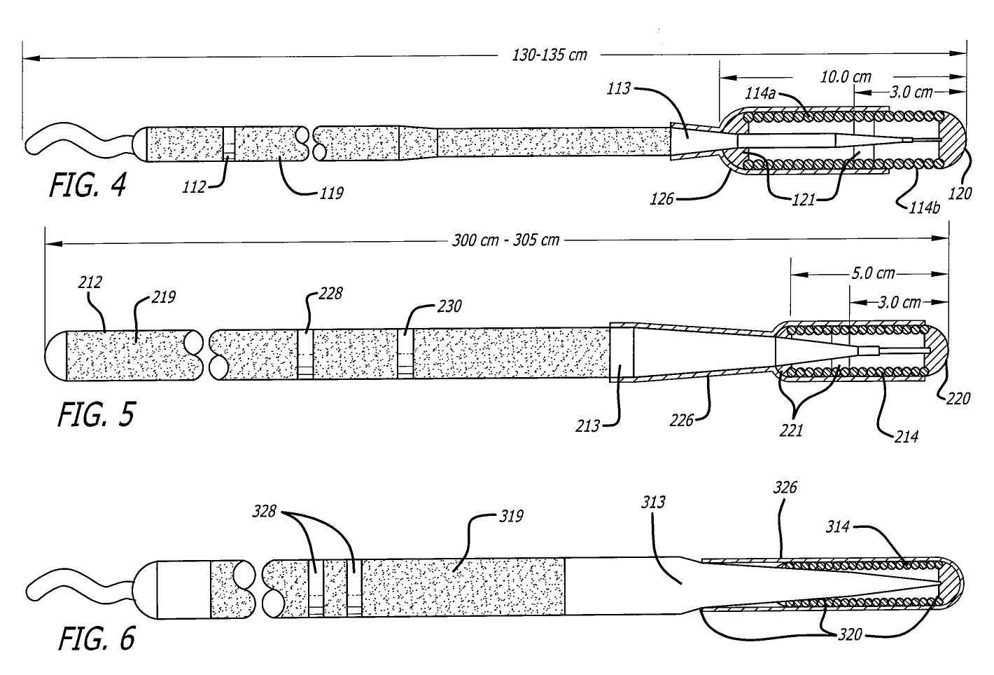

FIGS. 1-3 depict a guidewire 10 which has a core member 11 with a proximal core section 12, a distal core section 13 and a helical coil 14. The distal core section 12 has a first tapered segment 15 and a second tapered core segment 16 which is distally contiguous to the first tapered core segment. The second tapered segment 16 tapers at a greater degree than the first tapered segment and this additional taper provides a much smoother transition when the distal portion of the guidewire 10 is advanced through a tortuous passageway. The degree of taper of the first tapered core segment 15, i.e. the angle between the longitudinal axis 17 and a line tangent to the first tapered core segment 15 is about 2° to about 10°, whereas the taper of the second tapered core segment 16, i.e. the angle between the longitudinal axis and the second tapered core segment is larger than the first angle and is about 5° to about 10° such as is shown in the enlarged view of the guidewire 10 in FIG. 4. While only two tapered core segments are shown in the drawings, any number of tapered core segments can be employed. Moreover, all of a multiple of tapered core segments need not have increasing degrees of tapers in distal direction. However, two or more contiguous tapered core segments over a length of about 5 to 15 cm should have distally increasing degrees of tapering. The present invention relates in large part to a special addition to the lubricious coating 19, which is typically TEFLON®, by which the performance of the catheter is considerably improved. In particular, as will be discussed later on in this patent application, this special lubricious coating helps prevent the guidewire from locking up when entering the vascular system.

Typically, the first tapered segment is about 3 cm in length and the second tapered segment is about 4 cm in length. In a presently preferred embodiment, the guidewire 10 has a proximal core section 12 of about 0.014 inch (0.36 mm) in diameter, the first tapered core segment has a diameter ranging from 0.014 inch down to about 0.008 inch (0.36-0.20 mm) and the second tapered core segment has a diameter ranging from about 0.008 to about 0.002 inch (0.20-0.05 mm). A flattened distal tip 18 extends from the distal end of the second tapered core segment 16 to the body of solder 20 which secures the distal tip 18 of the core member 11 to the distal end of the helical coil 14. A body of solder 21 secures the proximal end of the helical coil 14 to an intermediate location on the second tapered segment 16.

The core member 11 is coated with a multilayer lubricious coating 19 (FIG. 1), comprised of layers 19a-c (FIG. 2). The base coat 19a (FIG. 2) may be any of a variety of coatings, such as a fluoropolymer, e.g. TEFLON® available from DuPont, and which extends the length of the proximal core section 12. These coatings on the proximal portion of the guidewire are discussed in greater detail below. The distal section 13 may also be provided with a coating, not shown for purposes of clarity, such as a hydrophilic coating of the type known in the art. A hydrophilic coating is typically preferred on the distal portion of the wire, as it comes into contact with the arterial wall.

The core member may be formed of stainless steel, NiTi alloys or combinations thereof such as described in U.S. Pat. No. 5,341,818 (Abrams et al) which has been incorporated herein. Other materials such as the high strength alloys described in U.S. Pat. No. 5,636,641 (Fariabi), which has also been incorporated herein by reference, may also be used.

The helical coil 14 is formed of a suitable radiopaque material such as platinum or alloys thereof or formed of other material such as stainless steel and coated with a radiopaque material such as gold. The wire from which the coil is made generally has a transverse diameter of about 0.003 inch (0.05 mm). The overall length of the coil 14 is typically about 3 cm. Multiple turns of the distal portion of coil 14 may be expanded to provide additional flexibility.

In an alternative embodiment shown in FIG. 5, the flattened distal segment of the core member shown in FIG. 1 is replaced with a Shaping ribbon 30 which is secured by its distal end to the distal end of the coil 14 and by its proximal end to the distal extremity of the core member 11.

While the specific embodiments described above are directed to tapered segments with constant tapers along their lengths, the taper need not be constant. For example, the tapers of contiguous core segments may be gradually increasing in the distal direction, with the taper, i.e. a tangent line, crossing the junction between the two adjacent tapers being a continuous function. Guidewires are generally about 90 to about 300 cm in length, and most commercially available guidewires for the coronary anatomy are either 175 cm or 190 cm in length.

Multiple tapers may be ground simultaneously or as separate operations. A centerless grinder with profile capabilities may be used to grind the tapers simultaneously. A manual centerless grinding may be employed to create separate tapers in separate operations. Tapers may also be formed by other means such as chemical means.

Considering now the lubricious coating 19 (FIG. 1), and its constituent layers 19a-c (FIG. 2), the proximal core section 12 is typically coated with a base coat 19a such as TEFLON®. However, to enhance the lubricious properties of the proximal portion of the guidewire, it has been discovered that adding one or more additional lubricious coatings on top of the lubricious coating 19a can significantly enhance the performance of the guidewire, by reducing resistance and preventing lock up. The one or more coatings atop the base coat 19a help to overcome the adhesion of clumps composed of blood, contrast or both.

In one embodiment, a hydrophobic, silicone-based coating 19b (FIG. 2) is placed atop the lubricious base coating 19a. This silicone coating may be, for example, comprised of the components of the Microglide® coating used commercially by Guidant Corporation. In one approach, the Microglide® coating is formed from two separate silicone-based layers, one atop the other (layers 19b and 19c in FIG. 2). In one formulation, the coating 19b is formed with a mixture of isopropyl alcohol, methylene chloride, and Dow Corning MDX-4-4159 silicone. The coating 19c is formed with a mixture of isopropyl alcohol, methylene chloride, and Dow 360 silicone. These dual silicone layers, applied sequentially atop the TEFLON® base coat, provide an especially slippery coating for outstanding performance in the body.

It is noted that the dual silicone formulations 19b and 19c may, alternatively, be mixed together prior to application. In that case, the components of the two formulations are applied as a single coat 19b that has advantages of both formulations. An additional coat of the mixed formulation may be applied as layer 19c or, alternatively, a single coat 19b atop the TEFLON® base coat may be sufficient.

In another approach, the silicone coating may be applied directly to the bare metal guide wire, which may optionally be pretreated to change its color. This direct-application approach takes advantage of the excellent adhesion of certain silicones to polar substrates such as bare steel. Furthermore, this eliminates the PTFE or polymer base coat process, thus making the wire easier and less costly to manufacture. Another advantage of this approach is that the overall diameter of the proximal section of the guide wire is now lessened by what would have been the thickness of the PTFE or polymer base-coat, allowing the guide wire to fit more easily into smaller diameter catheters.

As another variation, the silicone top coating or coatings may be applied to a base coat composed of a polymer or resin that has better silicone wetting and bonding characteristics as compared to TEFLON®. Examples include HDPE, surface treated HDPE or variations thereof.

In another embodiment in which the proximal portion of the guide wire is coated with a silicone-based coating, a preferred coating consists of amino functional dimethylsiloxane copolymer (Dow Corning MDX-4-4159 fluid, for example) and/or polydimethylsiloxane liquid (Dow 360, for example). The coating can range from 1-50% active silicone; however, the preferred embodiment would consist of a coating that is 2-10% active silicone. Substrates for the silicone-based coating could be, as alternatives to TEFLON®, various fluoropolymers, polyethylene, polypropylene, stainless steel, or nickel titanium alloys.

The coating is typically applied to the guidewire by dipping, wiping, spraying or any other method of application known in the art. The coating is oftentimes cured at room temperature, or may be heated (by, for example, blown heated air) to accelerate the cure. Optionally, the guidewire can be heated before applying the coating to help improve adhesion.

In another embodiment, the TEFLON®-coated proximal section is further coated with one or more additional layers of a release agent or compound The release layer, which can alternatively be called a “top” layer, can be from any of a number of release agent groups including, but not limited to, spray PTFE, paraffins, olefins, and fats.

In another embodiment, the proximal portion of the guide wire is coated with a polydimethylsiloxane liquid (Dow 360, for example). The coating can range in viscosity from 10 cs to 50,000 cs. However, the preferred embodiment would consist of a liquid with a viscosity of 100 cs to 1000 cs. Substrates for the polydimethylsiloxane coating could be fluoropolymers (including but not limited to polytetrafluoroethylene), polyethylene, polypropylene, stainless steel, or nickel titanium alloys. That is, the coating may be applied upon a TEFLON® base coat or, alternatively, upon another base coat or even upon bare metal.

In another embodiment, the silicone wax coating on the proximal portion of the guide wire is intended to provide performance benefits with regards to interaction between the catheter inner lumen and the guide wire because the silicone surface is resistant to the adhesion of blood and contrast.

The proximal portion of the guide wire is coated with a silicone wax coating. The coating consists of silicone wax with viscosities of 50,000 cP to 1,000,000 cP, with 75,000 cP to 100,000 cP being the preferred viscosity. Substrates for the silicone wax coating could be fluoropolymers (including but not limited to polyetrafluoroethylene), polyethylene, polypropylene, stainless steel, or nickel titanium alloys. The wire can be heated before applying the coating to help improve adhesion. The coating is applied to the wire with a wiping process. Heating the coating may allow for dip or spray processing.

The approach described thus far is not limited to a particular guidewire structure. The general principle in this approach is to make the proximal portion of the guidewire particularly lubricious, irrespective of the particular guidewire structure. The advantages of this approach can extend to a broad range of guidewire designs.

For example, FIG. 4 illustrates one of many possible alternative guidewire designs for use in peripheral areas of the body. The design of FIG. 4 includes a distal core section 112, a core wire sub-assembly 113, and both an intermediate coil 114a and a tip coil 114b. An area of solder 120 is present at the tip, and additional areas of solder 121 are present in regions of the distal portion of the guidewire. The proximal portion of the guidewire is coated with a lubricious coating 119, which may include a base coat of TEFLON® and followed by any of the top coats discussed previously. As with the previous design shown in FIGS. 1-3, the embodiment of FIG. 4 has a particularly lubricious coating on the proximal portion of the guidewire. It is noted that the distal portion of the guidewire may also be coated with a lubricious coating. Region 126 may be coated with a silicone-based coating such as Guidant Corporation's Microglide®. Such a coating on the distal portion allows the distal portion to slide easily through the anatomy. As an alternative to a Microglide® coating 126, a hydrophilic coating known in the art may be used instead.

FIG. 5 illustrates yet another guidewire embodiment in which the principles of the present invention are applied. A distal core section 212 leads to a core wire sub-assembly 213 and to a tip coil on the distal section 214. Areas of solder 220 and 221 are present in the distal tip. The distal core section 212 is typically coated with a lubricious coating 219 having a TEFLON® base and topped with a lubricious top coat as described previously. As with the other embodiments, a silicone-based top coat is preferably coated atop the TEFLON®. In yet another embodiment, two or more layers may be coated atop the TEFLON® layer, for a particularly lubricious coating.

Also shown in FIG. 5 are radiopaque markers 228 and 230. A proximal marker 228 is arranged proximally relative to a distal radiopaque marker 230. The proximal markers may be placed along the length of the guidewire, as desired.

FIG. 6 illustrates an advanced guidewire for use in the coronary region. A distal core section 312 is coated along the length thereof with a TEFLON® base coat topped with a silicone based or other top coat, as described with respect to FIGS. 1-3. The embodiment of FIG. 6 also includes a distal core wire 313, distal coils 314, and areas of solder 320. A hydrophilic coating 326 may be applied in the distal section. This hydrophilic coating 326 assists in performance of the distal portion of the guidewire as it works its way through the coronary artery system.

Considering further FIG. 6, a guidewire of this type is the HI-TORQUE ADVANCE™ family of guidewires from Guidant Corporation. The HI-TORQUE ADVANCE™ guidwires are well-suited for drug-eluting stents. They combine several Guidant technologies, such as the RESPONSEASE™ transitionless core, the DURASTEEL™ core-to-tip design, and SMOOTHGLIDE™ technology. The RESPONSEASE™ transitionless core grind provides excellent tracking and 1:1 torque response. The DURASTEEL™ high tensile strength core material provides durability and superb torque control. The core-to-tip design offers precise steering and tip control.

In this commercial family of guidewires, the distal portion of the wire is coated with a hydrocoat hydrophilic coating for, among other things, smooth lesion access. Tip and intermediate coils in the distal portion provide excellent tracking and tactile feedback. A transitionless parabolic core grind, as opposed to a conventional tapered core, eliminates prolapse points to provide easy tracking and conserves diameter to provide excellent torque response.

Generally, the HI-TORQUE ADVANCE™ family of guidewires are intended to facilitate the placement of interventional percutaneous transluminal coronary angioplasty (PTCA) and percutaneous transluminal angioplasty (PTA) catheters and other interventional devices including: intravascular stents, intravascular ultrasound devices and intravascular drug eluting stents.

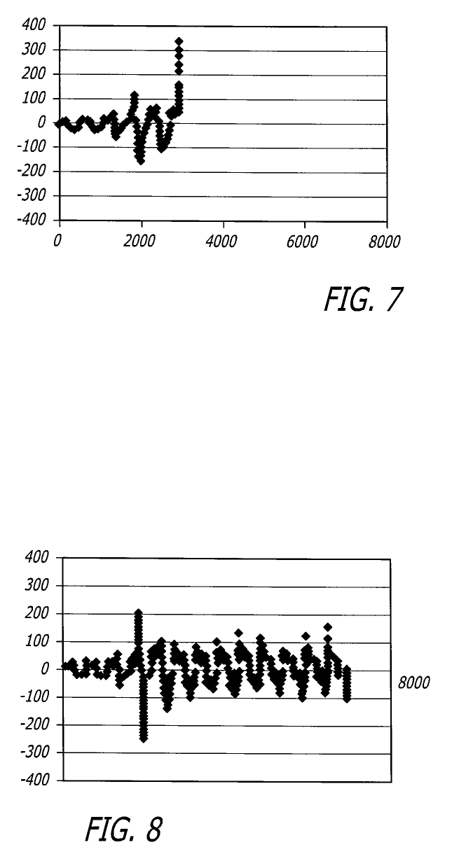

FIG. 7 illustrates a graph showing the relationship of force (along the vertical axis) versus displacement (along the horizontal axis) of a guidewire that is uncoated on the proximal portion. FIG. 7 generally reflects test results of a guidewire coated with sheep blood and contrast mixture, sliding back and forth within a catheter. The guidewire ultimately locks up, at what is shown in FIG. 7 at the far right-hand portion of the graph. At the point of lock-up, the catheter can no longer work its way through a vascular system without special manipulation.

FIG. 8, on the other hand, generally illustrates performance of a guidewire according to the present invention. The proximal portion of the guidewire is coated with a TEFLON® base and a silicone-based top coat. This particularly lubricious combination of coatings on the proximal portion of the guidewire yields a force-displacement response graph in which the catheter does not lock up, indicating a significant improvement over the uncoated guidewire of FIG. 7.

As a matter of terminology, the “proximal” portion of the guidewire is typically the lengthy segment wherein there is not a reduction in the diameter of the core of the wire. In contrast, the “distal” portion often extends from the tip of the wire for about 25-30 centimeters. During use, the “distal” portion travels outside of the guiding catheter and into the targeted portion of the anatomy, coming into contact with the arterial wall. While the “distal” portion is a minor length of the guidewire beginning at the tip, the “proximal” portion is the major part of the wire. As a non-limiting example, the “proximal” portion may extend for 160-270 centimeters in some guidewires.

As another matter of terminology, it is noted that in most preferred embodiments, the proximal portion is continuously coated with the lubricious coating except, in some embodiments, in areas that are specially designated as a radio-opaque marker. In this sense, “continuously coated” is meant to include instances in which the entire proximal portion is coated with the coating, and instances in which the entire proximal portion is coated except for radio-opaque markers. In other embodiments, however, the proximal portion may be pattern coated, with the lubricious coating applied in any pattern suitable for use in the body.

The term “TEFLON®” as used herein means polytetrafluoroethylene, or closely-related materials, such as fluorinated ethylene-propylene (FEP), perfluoroalkoxy polymer resin (PFA) and the like.

While particular forms of the invention have been illustrated and described, various modifications can be made without departing from the spirit and scope of the invention. Accordingly, it is not intended that the invention be limited, except as by the appended claims.

We claim:

1. A guidewire comprising:
a proximal portion; and
a distal portion;
wherein the proximal portion comprises a base coat of one lubricious coating and a top coat of a different lubricious coating.

2. A guidewire as defined in claim 1, wherein the base coat is a fluoropolymer.

3. A guidewire as defined in claim 1, wherein the base coat is polytetrafluoroethylene.

4. A guidewire as defined in claim 1, wherein the base coat is applied by at least one of dip coating, spraying and heat shrinking.

5. A guidewire as defined in claim 1, wherein the top coat is hydrophobic.

6. A guidewire as defined in claim 1, wherein the top coat comprises at least one silicone-based layer.

7. A guidewire as defined in claim 6, wherein the top coat comprises at least one curable silicone-based layer.

8. A guidewire as defined in claim 6, wherein the top coat comprises at least one non-curable silicone-based layer.

9. A guidewire as defined in claim 1, wherein the top coat comprises at least two silicone-based layers.

10. A guidewire as defined in claim 9, wherein a first layer of top coat is one silicone-based layer and the second layer of top coat is a different silicone-based layer.

11. A guidewire as defined in claim 1, wherein the top coat comprises at least one of the group constituting: a silicone-based coating, a paraffin, an olefin, and a fat.

12. A guidewire as defined in claim 1, wherein the distal portion is coated with a hydrophilic coating and the top coat of the proximal portion is a hydrophobic coating.

13. A guidewire as defined in claim 1, wherein the entire proximal region is coated with the base coat and the top coat.

14. A guidewire as defined in claim 1, wherein the entire base coated region is fully coated with the top coat.

15. A guidewire as defined in claim 1, wherein the entire base coated region is pattern coated with the top coat.

16. A guidewire as defined in claim 1, wherein the proximal region is pattern coated with the base coat and the top coat.

17. A guidewire comprising:
a proximal portion; and
a distal portion;
wherein the proximal portion is coated with a lubricious coating chosen from the group defined by: a silicone-based coating, a paraffin, an olefin, and/or a fat.

18. A guidewire as claimed in claim 17, wherein the coating on the proximal portion is coated onto bare metal.

19. A guidewire as claimed in claim 18, wherein the bare metal is specially tinted to indicate a specially-coated proximal region.

20. A guidewire as claimed in claim 17, wherein the coating is atop a base coat comprising at least one of the group consisting of: a fluoropolymer, polyethylene, and polypropylene.

21. A guidewire as claimed in claim 17, wherein the coating is continuous over the proximal region of the guidewire.

22. A guidewire as claimed in claim 17, wherein the coating is pattern coated on the proximal region of the guidewire.

23. A guidewire comprising:
a proximal portion; and
a distal portion comprising a guidewire tip;
wherein the proximal portion comprises a first base coat of lubricious coating and at least a one top coat on the base coat of a second, hydrophobic, lubricious coating; and
wherein the distal portion is coated with a hydrophilic coating.

24. A guidewire as defined in claim 23, wherein the base coat comprises a fluoropolymer.

25. A guidewire as defined in claim 23, wherein the base coat comprises polytetrafluoroethylene.

26. A guidewire as defined in claim 23, wherein the base coat is applied by at least one process selected from the group constituting: dip coating, spraying and heat shrinking.

27. A guidewire as defined in claim 23, wherein the top coat is at least one layer of silicone.

28. A guidewire as defined in claim 23, wherein the top coat comprises at least one curable silicone-based layer.

29. A guidewire as defined in claim 23, wherein the top coat comprises at least one non-curable silicone-based layer.

30. A guidewire as defined in claim 23, wherein the top coat is a curable silicone-based layer.

31. A guidewire as defined in claim 23, wherein the top coat is a non-curable silicone-based layer.

32. A guidewire as defined in claim 23, wherein the top coat comprises at least two silicone-based layers.

33. A guidewire as defined in claim 23, wherein a first layer of top coat is one silicone-based layer, and a second layer of top coat is a different silicone-based layer.

34. A guidewire as defined in claim 23, wherein the top coat comprises at least one of the group defined by: a silicone-based coating, a paraffin, an olefin, and a fat.

35. A guidewire as defined in claim 23, wherein the distal portion is coated with a hydrophilic coating, and the proximal portion is coated with hydrophobic coating.

36. A guidewire as defined in claim 23, wherein the proximal region is continuously coated with the base coat and the top coat over the entire proximal region.

37. A guidewire as defined in claim 23, wherein the entire base-coated region is coated with the top coat.

38. A guidewire as defined in claim 23, wherein the proximal region is pattern-coated with the base coat and top coat.

39. A guidewire as defined in claim 23, wherein the base coat is continuously coated on the proximal portion of the guidewire, and the top coat is pattern coated on the base coat.
